(19) United States
(12) Patent Application Publication
(10) Pub. No.: US 2014/0151252 A1
(43) Pub. Date: Jun. 5, 2014

(54) BAG FOR TRANSPORTING MEDICAL EQUIPMENT FOR AN INSULIN-DEPENDENT DIABETIC

(71) Applicant: Geoffroy Simon Billon, Amiens (FR)

(72) Inventor: Geoffroy Simon Billon, Amiens (FR)

(21) Appl. No.: 14/234,252

(22) PCT Filed: Jul. 26, 2012
(86) PCT No.: PCT/FR2012/051777

§ 371 Date: Jan. 22, 2014

(30) Foreign Application Priority Data
Jul. 26, 2011 (FR) 11/56787

(51) Int. Cl. A61M 5/00

(52) U.S. Cl. CPC A61M 5/003
USPC 206/366

(57) ABSTRACT

Disclosed is a transport bag including an enclosure that delimits at least a first thermally insulated compartment, which is designed to receive a refrigerated unit for the transport of insulin, a second thermally insulated compartment, which is designed to receive insulin pens, a third compartment, which is designed to receive a blood glucose meter and preferably accessories of the blood glucose meter, and a first box and a second box which are fitted inside the enclosure, the first and second boxes being intended to hold new and used needles, respectively.

The present invention relates to a bag for transporting medical equipment for an insulin-dependent diabetic.

A bag for transporting medical equipment for an insulin-dependent diabetic known from the prior art includes several flexible pockets wherein medical equipment is arranged. Such a bag in the prior art is not entirely satisfactory as far as:
it does not allow to store all the medical equipment needed for several days of treatment,
it does not allow to manage differentiated storage for new and used needles,
needles may become detached from the pockets and lead to the risk of being pricked during a subsequent opening of the bag.

The purpose of the present invention is to overcome in whole or in part the aforementioned disadvantages and relates to a bag for transporting medical equipment for insulin-dependent diabetic, the carrying bag being characterized in that it comprises:
an enclosure delimiting at least:
   a first thermally insulated compartment, and adapted to receive a refrigerated unit for the transport of insulin,
   a second thermally insulated compartment, the second compartment being adapted to receive insulin pens,
   a third compartment adapted to receive a blood glucose meter and preferably blood glucose meter accessories,
a first casing and a second casing mounted inside the enclosure, the first and second casings being intended to hold new and used needles, respectively.

Thus, such a carrying bag according to the invention makes it possible to store the equivalent of several days, typically eight days, of medical equipment within the same enclosure.

Furthermore, such a bag makes it possible to effectively protect the needles during transport by means of the first and second casings enclosing said needles.

Moreover, the first and second casings make it possible to separate new and used needles.

In one embodiment, the first and second casings are removably mounted inside the enclosure.

Thus, it is possible to easily remove the needles from the first and second casings, without handling the carrying bag. It is particularly possible to easily remove the used needles from the second casing without the risk of being pricked.

According to one embodiment, the enclosure comprises means for magnetization, and the first and second casings each comprise complementary magnetization means arranged to cooperate with the means of magnetization by opposite magnetic poles to removably fix the first and second casings within the enclosure.

In one embodiment, the first and second casings are each fitted with a double zip system.

Thus, such a double zip system makes it possible to provide a large opening while allowing for an easy filling.

Advantageously, the first and second casings each has a textile sheath wherein reinforcing members are arranged, preferably rigid plastic sheets.

Thus, such reinforcing members make it possible to prevent the textile sheaths from being pierced by the needles and the user from being pricked.

Advantageously, the enclosure delimits a fourth compartment adapted to receive the first and second substantially juxtaposed casings.

Thus, it is possible to obtain a more compact carrying bag.

According to one feature, the carrying bag comprises a bottom wall and at least one side wall extending from the bottom wall such as to form the enclosure.

Preferentially, the bottom wall and/or each side wall have a textile sheath wherein reinforcing members are arranged, preferably rigid plastic sheets.

Thus, the medical equipment is protected and the risk of perforating the textile sheaths is highly limited.

In one embodiment, the carrying bag comprises a first movable flap between a closed position wherein it blocks the enclosure, and an open position wherein it frees access to the enclosure, the locking of the first flap in the closed position being preferably obtained using a zip system.

Preferentially, the first flap has a textile sheath wherein reinforcing members are arranged, preferably rigid plastic sheets.

Thus, such a first flap helps protect the medical equipment.

According to one embodiment, the carrying bag comprises a second flap removably mounted on the first flap and/or the enclosure, the second flap being provided with a storage pocket and a mesh pocket preferably fitted with a zip system.

Thus, such a second flap makes it possible to receive the medical equipment for an insulin-dependent diabetic for one day.

Other features and advantages will become apparent in the following description of an embodiment of a carrying bag according to the invention, given by way of a non-limiting example, with reference to the accompanying drawings in which:

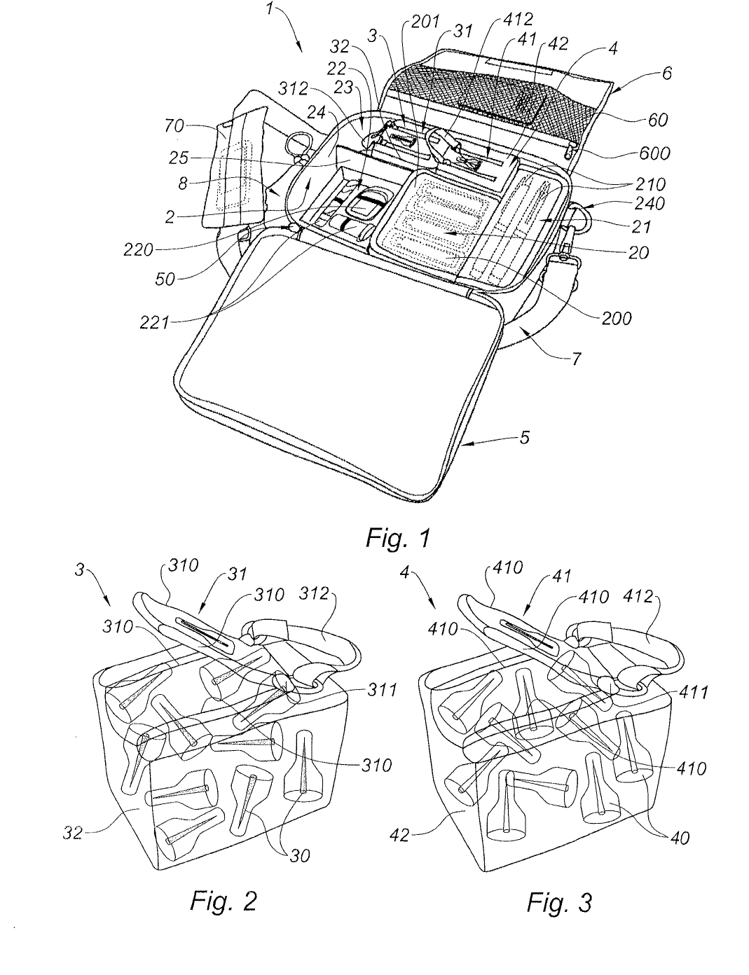

FIG. 1 is a perspective view of a carrying bag according to the invention including cutaways of medical equipment elements present in said bag,

FIGS. 2 and 3 are magnified perspective views of the first and second casings representing in transparency new and used needles respectively.

The bag for transporting medical equipment 1 for an insulin-dependent diabetic shown in FIG. 1 comprises an enclosure 2 delimiting:
a first thermally insulated compartment 20, and adapted to receive a refrigerated unit 200 (represented in transparency by dotted lines) for the transport of insulin in good conditions for a few hours,
a second thermally insulated compartment 21, the second compartment 21 being adapted to receive insulin pens 210 (represented in transparency by dotted lines) or insulin cartridges (not shown); the insulin pens 210 may be disposable or refillable,
a third compartment 22 adapted to receive a blood glucose meter 220 and meter accessories 221, said accessories 221 can be a pouch, a lancet, needles or even strips.

The first compartment 20 is fitted with a top 201 fashioned to block the first compartment 20. Likewise, the second compartment 21 is fitted with a top 202 fashioned to block the second compartment 21. The second compartment 21 comprises at least one strip made from elastic material, the strip comprising at least one loop fashioned to receive an insulin pen 210.

The carrying bag 1 further comprises a first casing 3 and a second casing 4 mounted inside the enclosure 1, the first and second casings 3, 4 being intended to enclose respectively, new needles 30 and used needles 40. The new and used needles 30, 40 are shown in FIGS. 2 and 3.

The first and second casings 3, 4 are removably mounted inside the enclosure 2. To this end, the enclosure 1 comprises means of magnetization such as a permanent type magnet (not shown). The first and second casings 3, 4 each comprise complementary magnetization means, such as a permanent type magnet (not shown), arranged to cooperate with the magnetization means of the enclosure 1 by opposite magnetic poles to removably fix the first and second casings 3, 4 inside the enclosure 1.

The first and second bags 3, 4 are of substantially parallelepiped shape. The first and second casings 3, 4 each have a textile sheath 32, 42 wherein reinforcing members (not shown) are arranged, preferably rigid plastic sheets. The textile sheath may be made of washable polyester.

The first and second casings 3, 4 are equipped with identification means, such as a pictogram designed to identify new and used needles.

The first and second casings 3, 4 are each equipped with a double zip system 31, 41 arranged on the upper side of the first and second casings 3, 4.

Each double zip system 31, 41 comprises:
two fabric strips 310, 410 fitted with teeth,
a runner 311, 411 fashioned to encase or separate the teeth.

The fabric strips 310, 410 of each double zip system 31, 41 are arranged on the upper side of the first and second casings 3, 4 such as to delimit a substantially rectangular area.

The runners 311, 411 of each double zip system 31, 41 are interconnected by a strap 312, 412 such as to facilitate the opening/closure of the corresponding casing 3, 4.

Furthermore, the enclosure 2 delimits a fourth compartment 23 adapted to receive the substantially juxtaposed first and second casings 3, 4.

The enclosure 2 comprises a bottom wall (not visible) and a side wall 24 extending from the bottom wall such as to form the enclosure 2. The bottom wall and the side wall 24 each exhibit a textile sheath wherein reinforcing members are arranged, preferably rigid plastic sheets.

The enclosure 2 is provided with partition walls 25 (only one is visible) making it possible to delimit compartments 20, 21, 22, 23.

The carrying bag 1 includes a first movable flap 5 between a closed position wherein it blocks the enclosure 2, and an open position (shown in FIG. 1) wherein it releases access to the enclosure 2, the first flap 5 being lockable into a closed position using a zip system 50.

The first flap 5 has a textile sheath 5 wherein reinforcing members are arranged, preferably rigid plastic sheets.

The carrying bag 1 comprises a second flap 6 removably mounted on the side wall 24 of the enclosure 2 and on the first flap 5, for example using velcro type strips.

The second flap 6 is equipped with a storage pocket and a mesh pocket 60 equipped with a zip system 600.

Furthermore, the carrying bag 1 comprises a shoulder strap 7 removably mounted on the fastening eyelets 240 fitted to the side wall 24 of the enclosure 2. The shoulder strap 7 comprises a pouch 70 adapted to receive an apparatus such as a mobile phone.

Furthermore, the carrying bag 1 comprises a carrying handle (not shown) able to be fixed to the second flap 6.

The carrying bag 1 also comprises a drink holder 8 arranged on the side wall 24 of the enclosure 2. The drink holder 8 can be achieved in the form of a mesh pocket.

Of course, the embodiment of the aforementioned invention is in no way limiting. Details and improvements can be made in other alternative embodiments without departing from the scope of the invention.

1. A carrying bag for medical equipment for an insulin-dependent diabetic, wherein the carrying bag comprises:
enclosure delimiting at least:
a first thermally insulated compartment, and adapted to receive a refrigerated unit for the transport of insulin,
a second thermally insulated compartment, the second compartment being adapted to receive insulin pens,
a third compartment adapted to receive a blood glucose meter and preferably blood glucose meter accessories,
a first casing and a second casing mounted inside the enclosure, the first and second casings being intended to hold new and used needles, respectively.

2. The carrying bag according to claim 1, wherein the first and second casings are removable mounted within the enclosure.

3. The carrying bag according to claim 2, wherein the enclosure comprises magnetization means, and in that the first and second casings each comprises complementary magnetization means arranged to cooperate with the magnetization means by opposite magnetic poles in order to removably first the first and second casings within the enclosure.

4. The carrying bag according to claim 1, wherein the first and second casings are each equipped with the double zip system.

5. The carrying bag according to claim 1, wherein the first and second casings each exhibit a textile sheath wherein reinforcing members are arranged, preferably rigid plastic sheets.

6. The carrying bag according to claim 1, wherein the enclosure delimits a fourth compartment adapted to receive the substantially juxtaposed first and second casings.

7. The carrying bag according to claim 1, wherein the carrying bag comprises a bottom wall and at least one side wall extending from the bottom wall such as to form the enclosure.

8. The carrying bag according to claim 7, wherein the bottom wall and/or each side wall have a textile sheath wherein reinforcing members are arranged, preferably rigid plastic sheets.

9. The carrying bag according to claim 6, wherein the carrying bag comprises a first flap movable between a closed position wherein it blocks the enclosure, and an open position wherein it frees access to the enclosure, the first flap being preferably locked in the closed position using a zip system.

10. The carrying bag according to claim 9, wherein the carrying bag comprises a second flap removably mounted on the first flap and/or the enclosure, the second flap being equipped with a storage pocket and a mesh pocket preferably equipped with a zip system.
